(19) United States
(12) Patent Application Publication
(10) Pub. No.: US 2023/0293160 A1
(43) Pub. Date: Sep. 21, 2023

(54) SURGICAL INSTRUMENT WRIST

(71) Applicant: Intuitive Surgical Operations, Inc., Sunnyvale, CA (US)

(72) Inventor: Andrew C. WATERBURY, Santa Clara, CA (US)

(73) Assignee: Intuitive Surgical Operations, Inc., Sunnyvale, CA (US)

(21) Appl. No.: 18/101,503

(22) Filed: Jan. 25, 2023

Related U.S. Application Data
(60) Provisional application No. 63/303,600, filed on Jan. 27, 2022.

(51) Int. Cl. A61B 17/00; A61B 34/35

(52) U.S. Cl. CPC A61B 17/00234; A61B 34/35; A61B 2017/00323; A61B 2017/00477

(57) ABSTRACT

A medical device includes a wrist link, an inner pulley, an outer pulley support, an outer pulley, a first tension member, and a second tension member. The wrist list includes a wrist link body and an inner pulley support extending outward from the wrist link body. The inner pulley is rotatably mounted on the inner pulley support and the inner pulley support extending a first distance away from the wrist link. The outer pulley support is coupled to the wrist link body and extends spaced from the wrist link body. The outer pulley is rotatably mounted on the outer pulley support. The outer pulley support is spaced a second distance away from the wrist link, and the second distance is larger than the first distance. The first tension member extends around the inner pulley and the second tension member extends around the outer pulley.

### CROSS-REFERENCE TO RELATED APPLICATIONS

This application claims priority to and the benefit of U.S. Provisional Application No. 63/303,600, entitled “SURGICAL INSTRUMENT WRIST” filed Jan. 27, 2022, which is incorporated herein by reference in its entirety.

### BACKGROUND

The embodiments described herein relate to grasping tools, and more specifically to medical devices, and still more specifically to endoscopic tools. More particularly, the embodiments described herein relate to devices that include tension cables and mechanisms for guiding the tension cables through the wrist link members. More particularly, the embodiments described herein relate to devices that include guide mechanisms that can be used, for example, in surgical applications.

Known techniques for Minimally Invasive Surgery (MIS) employ instruments to manipulate tissue that can be either manually controlled or controlled via computer-assisted teleoperation. Many known MIS instruments include a therapeutic or diagnostic end effector (e.g., forceps, a cutting tool, or a cauterizing tool) mounted on a wrist mechanism at the distal end of a shaft. During an MIS procedure, the end effector, wrist mechanism, and the distal end of the shaft can be inserted into a small incision or a natural orifice of a patient to position the end effector at a work site within the patient’s body. The optional wrist mechanism can be used to change the end effector’s orientation with respect to the shaft to perform the desired procedure at the work site. In known instruments, motion of the instrument as a whole provides mechanical degrees of freedom (DOFs) for movement of the end effector and the wrist mechanisms generally provide the desired DOFs for movement of the end effector with reference to the shaft of the instrument. For example, for forceps or other grasping tools, known wrist mechanisms are able to change the pitch and yaw of the end effector with reference to the shaft. A wrist may optionally provide a roll DOF for the end effector, or the roll DOF may be implemented by rolling the shaft. An end effector may optionally have additional mechanical DOFs, such as grip or knife blade motion. In some instances, wrist and end effector mechanical DOFs may be combined. For example, U.S. Pat. No. 5,792,135 (filed May 16, 1997) discloses a mechanism in which wrist and end effector grip DOFs are combined.

To enable the desired movement of the wrist mechanism and end effector, known instruments include mechanical connectors (e.g., cables) that extend through the shaft of the instrument and that connect the wrist mechanism to a mechanical structure configured to move the cables to operate the wrist mechanism and end effector. For telesurgical systems, the mechanical structure is typically motor driven and can be operably coupled to a computer processing system to provide a user interface for a clinical user (e.g., a surgeon) to control the instrument as a whole, as well as the instrument’s components and functions.

Patients benefit from continual efforts to improve the effectiveness of MIS methods and tools. For example, reducing the size and/or the operating footprint of the shaft and wrist mechanism can allow for smaller entry incisions and reduced need for space at the surgical site, thereby reducing the negative effects of surgery, such as pain, scarring, and undesirable healing time. But, producing small medical instruments that implement the clinically desired functions for minimally invasive procedures can be challenging. Specifically, simply reducing the size of known wrist mechanisms by “scaling down” the components will not result in an effective solution because required component and material properties do not scale in part because the mechanical advantage decreases, but the surgical site forces remain same for a given task. For example, efficient implementation of a wrist mechanism can be complicated because the cables must be carefully routed through the wrist mechanism to maintain cable tension throughout the range of motion of the wrist mechanism and to minimize the interactions (or coupling effects) of one rotation axis upon another. Further, pulleys and/or contoured surfaces are generally needed to reduce cable friction, which extends instrument life and permits operation without excessive forces being applied to the cables or other structures in the wrist mechanism. Increased localized forces that may result from smaller structures (including the cables and other components of the wrist mechanism) can result in undesirable stress or wear of cables during cleaning and use, reduced cable life, and the like.

Further, some medical instruments have end effectors that require electrical energy for clinical functions such as desiccation, hemostasis, cutting, dissection, fulguration, incisions, tissue destruction, cauterizing, and vessel sealing. Accordingly, known instruments include one more conductors routed through the wrist mechanism to the portion of an end effector to be energized. Fitting all the components of the wrist mechanism, drive cables, and conductive wires into a small diameter, for example, less than about 8.5 mm while preserving the necessary strength and function of these components can be difficult.

In instances where the instrument size is scaled down, it is also desirable to maintain or improve the cable cycle life (cycles to cable break) of the scaled down cable path. By maintaining or improving cable cycle life, the instruments can be used and re-used in multiple surgical procedures.

Thus, a need exists for improved endoscopic tools, including improved wrist mechanisms having reduced size and a pulley support arrangement that scales down while being able to transfer sufficient force to end effectors without negatively impacting overall cycle life of the cables.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

In some embodiments, a medical device includes a wrist link, an inner pulley, an outer pulley, a first tension member, and a second tension member. The wrist link includes a wrist link body and an inner pulley support extending outward from the wrist link body. The inner pulley is rotatably mounted on the inner pulley support to rotate about an inner pulley axis at a first distance from the wrist link. The outer pulley support member is coupled to the wrist link body and extends toward the wrist link body. The outer pulley is rotatably mounted on the outer pulley support to rotate about an outer pulley axis at a second distance from the wrist link, the second distance being larger than the first distance. The first tension member extends around the inner pulley and the second tension member extends around the outer pulley.

In some embodiments, the medical device includes an outer pulley support bracket. The outer pulley support bracket includes a mounting portion that extends between the outer pulley support and the wrist link body. In some embodiments, the outer pulley axis of rotation does not intersect the inner pulley support.

In some embodiments, the medical device includes an outer pulley support bracket. The outer pulley support bracket includes a mounting portion that extends between the outer pulley support and the inner pulley support.

In some embodiments, the medical device includes an outer pulley support bracket. The outer pulley support bracket includes a first mounting portion and a second mounting portion. The first mounting portion extends between the outer pulley support and the wrist link body. The second mounting portion extends between the outer pulley support and the inner pulley support.

In some embodiments, the first tension member is routed about the inner pulley along an inner pulley arc length. The second tension member is routed about the outer pulley along an outer pulley arc length smaller than the inner pulley arc length.

In some embodiments, the outer pulley axis of rotation is parallel to the inner pulley axis of rotation.

In some embodiments, the inner pulley includes an outer perimeter and the outer pulley includes an outer perimeter. A projection of the outer perimeter of the outer pulley parallel to the outer pulley axis of rotation overlaps the outer perimeter of the inner pulley.

In some embodiments, the inner pulley includes an outer perimeter. The outer pulley axis of rotation is outside a projection of the outer perimeter of the inner pulley parallel to the inner pulley axis of rotation.

In some embodiments, the wrist link body includes a first material and the outer pulley support bracket comprises a second material. The second material is different from the first material.

In some embodiments, the inner pulley and the outer pulley are enclosed between the wrist link and the outer pulley support bracket.

In some embodiments, the inner pulley has a circumference defined by an outer radius of the inner pulley. The first tension member is routed about a portion of the circumference of the inner pulley, and the first tension member has a cross-sectional radius. A ratio of the outer radius of the inner pulley to the cross-sectional radius of the first tension member is between about 6.5 to 12.

In some embodiments, the first and second tension members are tungsten cables.

In some embodiments, the wrist link is sized to be inserted through a cannula having an inner diameter equal to or smaller than about 8.5 mm.

In some embodiments, the medical device includes a first tool member and a second tool member. The first and second tool members are rotatably coupled to the wrist link. The first tension member is coupled to the first tool member, and the second tension member is coupled to the second tool member.

In some embodiments, the inner pulley support extends to the outer pulley support bracket.

In some embodiments, the inner pulley support extends outside of a projection of an outer perimeter of the outer pulley parallel to the outer pulley axis of rotation.

In some embodiments, the outer pulley support bracket is coupled to the wrist link body in a snap-fit configuration.

In some embodiments, the outer pulley support bracket is coupled to the wrist link body in a friction fit configuration.

In some embodiments, the medical device includes a teleoperated surgical instrument. The teleoperated surgical instrument includes the wrist link, the inner pulley, the outer pulley, the outer pulley support, the first tension member, and the second tension member.

Other medical devices, related components, medical device systems, and/or methods according to embodiments will be or become apparent to one with skill in the art upon review of the following drawings and detailed description. It is intended that all such additional medical devices, related components, medical device systems, and/or methods included within this description be within the scope of this disclosure.

### BRIEF DESCRIPTION OF THE DRAWINGS

FIG. 1 is a plan view of a minimally invasive teleoperated surgery system according to an embodiment being used to perform a medical procedure such as surgery.

FIG. 2 is a perspective view of an optional auxiliary unit of the minimally invasive teleoperated surgery system shown in FIG. 1.

FIG. 3 is a perspective view of a user control console of the minimally invasive teleoperated surgery system shown in FIG. 1.

FIG. 4 is a front view of a manipulator unit, including a plurality of instruments, of the minimally invasive teleoperated surgery system shown in FIG. 1.

FIG. 5 is a diagrammatic illustration of a portion of a medical instrument according to an embodiment.

FIG. 6 is a diagrammatic illustration of the portion of the medical instrument of FIG. 5 with an outer support structure hidden to show the routing of the drive cables.

FIG. 7 is a cross-sectional view of the portion of the medical instrument taken at cutting plane line A—A in FIG. 5.

FIG. 8 is a cross-sectional view of a portion of a medical instrument according to an embodiment.

FIG. 9 is a cross-sectional view of a portion of a medical instrument according to an embodiment.

FIG. 10 is a front perspective view of a portion of a medical instrument according to an embodiment.

FIG. 11 is a rear perspective view of the portion of the medical instrument in FIG. 10.

FIG. 12 is a side view of the portion of the medical instrument in FIG. 10.

FIG. 13 is a front side view of the portion of the medical instrument in FIG. 10 with the support bracket and proximal link hidden.

FIG. 14 is a rear side view of the portion of the medical instrument in FIG. 10 with the support bracket and proximal link hidden.

FIG. 15 is a front perspective view of the portion of the medical instrument with the support bracket hidden.

FIG. 16 is a front perspective view of the portion of the medical instrument with the support bracket and idler pulleys hidden.

FIG. 17 is a cross-sectional front perspective view of the portion of the medical instrument of FIG. 12 taken at cutting plane line 17—17.

FIG. 18 is a cross-sectional front perspective view of the portion of the medical instrument of FIG. 12 taken at cutting plane line 18—18.

FIG. 19 is a cross-sectional front elevation view of the portion of the medical instrument of FIG. 12 taken at cutting plane line 18—18.

FIG. 20 is a front perspective view of a support bracket according to an embodiment.

FIG. 21 is a rear perspective view of the support bracket of FIG. 19.

### DETAILED DESCRIPTION

The embodiments described herein can advantageously be used in a wide variety of grasping, cutting, and manipulating operations associated with minimally invasive surgery. The embodiments described herein can also be used in a variety of non-medical applications such as, for example, teleoperated systems for search and rescue, remotely controlled submersible devices, aerial devices, and automobiles, etc. The medical instruments or devices of the present application enable motion in three or more degrees of freedom (DOFs). For example, in some embodiments, an end effector of the medical instrument can move with reference to the main body of the instrument in three mechanical DOFs, e.g., pitch, yaw, and roll (shaft roll). There may also be one or more mechanical DOFs in the end effector itself, e.g., two jaws, each rotating with reference to a clevis (2 DOFs) and a distal clevis that rotates with reference to a proximal clevis (one DOF). Thus, in some embodiments, the medical instruments or devices of the present application enable end effector motion in all six Cartesian DOFs, with optional additional mechanical or control DOFs for other end effector functions. such as moving one jaw in opposition to another jaw. In other embodiments, instrument end effector motion in one or more Cartesian DOFs may be restricted. The embodiments described herein enable further miniaturization of the wrist and shaft assemblies to promote MIS procedure.

The medical instruments described herein can include narrow cables (e.g., cables with a cross-sectional diameter of about 0.457 mm (0.018 inch) to about 0.635 mm (0.025 inch)) that are guided by corresponding pulley members. The pulley members (i.e., an inner pulley member and an outer pulley member) are offset both laterally and axially to route the narrow cables in a manner which reduces stress and improves cable cycle life (i.e., the number of operational cycles before the cable will break). Specifically, by routing the cables in a manner that reduces stress, the cables will be able to undergo a great number of tension cycles before reaching a theoretical breaking point. Accordingly, the embodiments described herein can allow for a great number of cycles (i.e., uses) for the instrument must be taken out of service. To achieve the lateral and axial offset of the pulley members, a separate mounting structure is provided to support the outer pulley member such that the rotational portion of the outer pulley member overlaps with a support member (i.e., axle) of the inner pulley member, and the rotational portion of the inner pulley member overlaps with a support member (i.e., axle) of the outer pulley member. The separate mounting structure enables the inner pulley and outer pulley to be optimally positioned relative to the driven pulleys of the end effectors, while also keeping the overall medical instrument compact (e.g., maintaining an overall cross-sectional diameter between about 4.0 mm and 10 mm (less than about 10 mm, and preferably with an overall cross-sectional diameter of less than about 8.5 mm), and more preferably with an overall cross-sectional diameter of between about 4.0 mm and 6.0 mm, and more preferably with an overall cross-sectional diameter of about 5.0 mm).

Additionally, the instruments described herein can include a tool member (e.g., a grasper, blade, etc.) that include jaws. Each of the jaws are coupled to a corresponding drive pulley that is offset from one another along a rotation axis of the tool member. Cables (which function as tension members) can be wrapped about the drive pulleys. Movement of the cables can cause each jaw to move independently or in concert with one another (e.g., to open jaws, to close the jaws, or to move both jaw members in a same direction about the rotation axis).

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

The term “flexible” in association with a part, such as a mechanical structure, component, or component assembly, should be broadly construed. In essence, the term means the part can be repeatedly bent and restored to an original shape without harm to the part. Certain flexible components can also be resilient. For example, a component (e.g., a flexure) is said to be resilient if possesses the ability to absorb energy when it is deformed elastically, and then release the stored energy upon unloading (i.e., returning to its original state). Many “rigid” objects have a slight inherent resilient “bendiness” due to material properties, although such objects are not considered “flexible” as the term is used herein.

As used in this specification and the appended claims, the word “distal” refers to direction towards a work site, and the word “proximal” refers to a direction away from the work site. Thus, for example, the end of a tool that is closest to the target tissue would be the distal end of the tool, and the end opposite the distal end (i.e., the end manipulated by the user or coupled to the actuation shaft) would be the proximal end of the tool.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial device positions and orientations. The combination of a body’s position and orientation define the body’s pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, instrument, and variants thereof, can be interchangeably used.

Aspects of the invention are described primarily in terms of an implementation using a da Vinci® Surgical System, commercialized by Intuitive Surgical, Inc. of Sunnyvale, California. Examples of such surgical systems are the da Vinci Xi® surgical system (Model IS4000), da Vinci X® surgical system (Model IS4200), and the da Vinci Si® surgical system (Model IS3000). Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations on da Vinci® surgical systems (e.g., the Model IS4000, the Model IS3000, the Model IS2000, the Model IS1200, the Model SP1099) are merely presented as examples, and they are not to be considered as limiting the scope of the inventive aspects disclosed herein. As applicable, inventive aspects may be embodied and implemented in both relatively smaller, hand-held, hand-operated devices and relatively larger systems that have additional mechanical support—i.e., on devices that are either mechanically grounded or ungrounded with reference to a world reference frame.

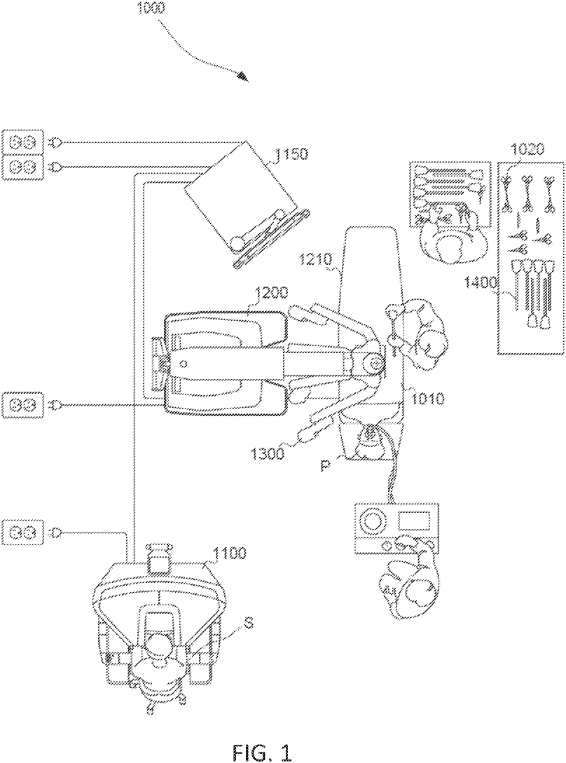

FIG. 1 is a plan view illustration of a computer-assisted teleoperation system. Shown is a medical device, which is a Minimally Invasive Robotic Surgical (MIRS) system 1000 (also referred to herein as a minimally invasive teleoperated surgery system that operates with at least partial computer support - a telesurgical system), used for performing a minimally invasive diagnostic or surgical procedure on a Patient P who is lying on an Operating table 1010. The system can have any number of components, such as a user control unit 1100 for use by a surgeon or other skilled clinician S during the procedure. The MIRS system 1000 can further include a manipulator unit 1200 (popularly referred to as a surgical robot), and an optional auxiliary equipment unit 1150. The manipulator unit 1200 can include an arm assembly 1300 and a tool assembly removably coupled to the arm assembly. The manipulator unit 1200 can manipulate at least one removably coupled instrument 1400 through a minimally invasive incision in the body or natural orifice of the patient P while the surgeon S views the surgical site and controls movement of the instrument 1400 through control unit 1100. An image of the surgical site is obtained by an endoscope (not shown), such as a stereoscopic endoscope, which can be manipulated by the manipulator unit 1200 to orient the endoscope. The auxiliary equipment unit 1150 can be used to process the images of the surgical site for subsequent display to the Surgeon S through the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces it with another instrument 1400 from a tray 1020 in the operating room. Although shown as being used with the instruments 1400, any of the instruments described herein can be used with the MIRS 1000.

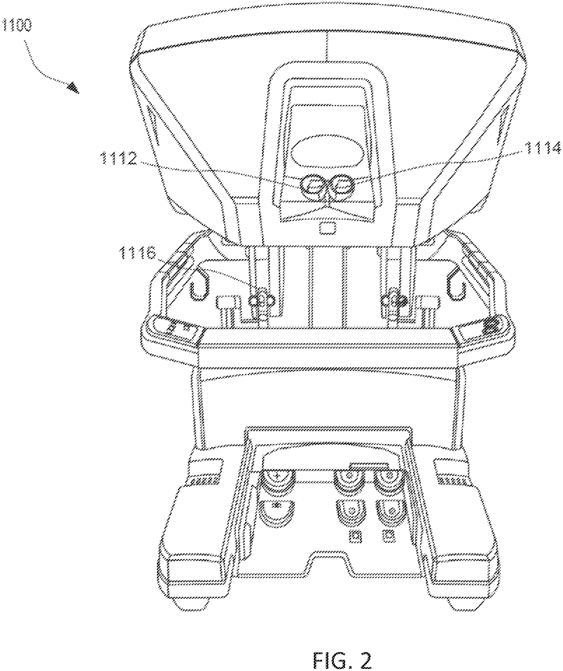

FIG. 2 is a perspective view of the control unit 1100. The user control unit 1100 includes a left eye display 1112 and a right eye display 1114 for presenting the surgeon S with a coordinated stereo view of the surgical site that enables depth perception. The user control unit 1100 further includes one or more input control devices 1116, which in turn cause the manipulator unit 1200 (shown in FIG. 1) to manipulate one or more tools. The input control devices 1116 provide at least the same degrees of freedom as instruments 1400 with which they are associated to provide the surgeon S with telepresence, or the perception that the input control devices 1116 are integral with (or are directly connected to) the instruments 1400. In this manner, the user control unit 1100 provides the surgeon S with a strong sense of directly controlling the instruments 1400. To this end, position, force, and tactile feedback sensors (not shown) may be employed to transmit position, force, and tactile sensations from the instruments 1400 back to the surgeon’s hands through the input control devices 1116.

The user control unit 1100 is shown in FIG. 1 as being in the same room as the patient so that the surgeon S can directly monitor the procedure, be physically present if necessary, and speak to an assistant directly rather than over the telephone or other communication medium. In other embodiments however, the user control unit 1100 and the surgeon S can be in a different room, a completely different building, or other remote location from the patient allowing for remote surgical procedures.

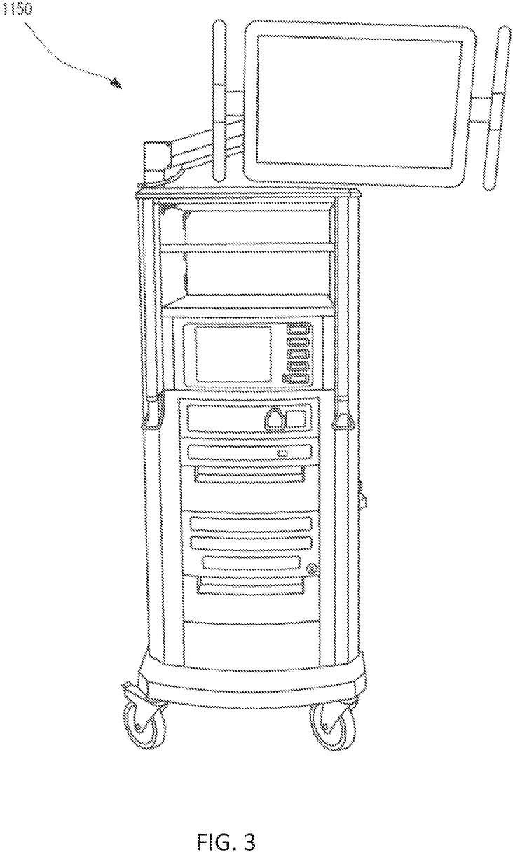

FIG. 3 is a perspective view of the auxiliary equipment unit 1150. The auxiliary equipment unit 1150 can be coupled with the endoscope (not shown) and can include one or more processors to process captured images for subsequent display, such as via the user control unit 1100, or on another suitable display located locally and/or remotely. For example, where a stereoscopic endoscope is used, the auxiliary equipment unit 1150 can process the captured images to present the surgeon S with coordinated stereo images of the surgical site via the left eye display 1112 and the right eye display 1114. Such coordination can include alignment between the opposing images and can include adjusting the stereo working distance of the stereoscopic endoscope. As another example, image processing can include the use of previously determined camera calibration parameters to compensate for imaging errors of the image capture device, such as optical aberrations.

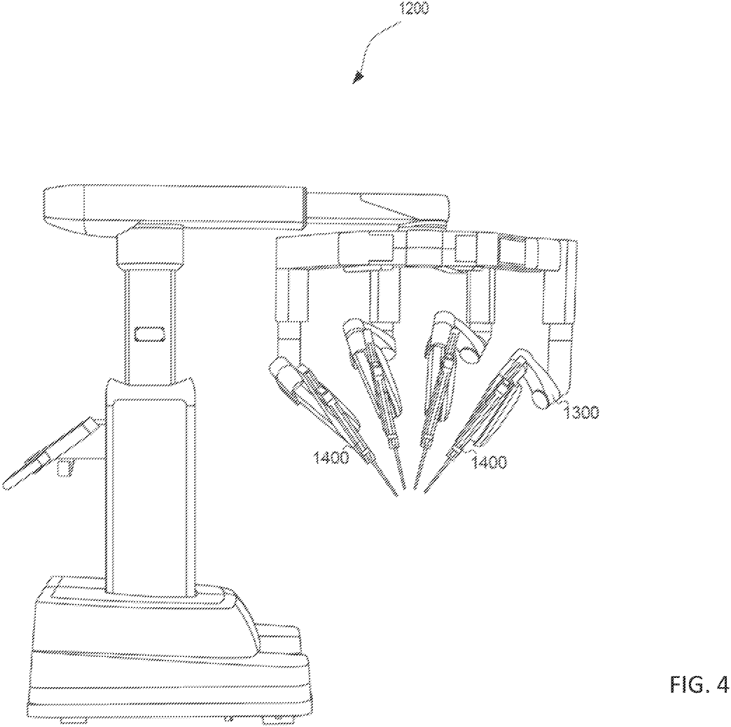

FIG. 4 shows a front perspective view of the manipulator unit 1200. The manipulator unit 1200 includes the components (e.g., arms, linkages, motors, sensors, and the like) to provide for the manipulation of the instruments 1400 and an imaging device (not shown), such as a stereoscopic endoscope, used for the capture of images of the site of the procedure. Specifically, the instruments 1400 and the imaging device can be manipulated by teleoperated mechanisms having a number of joints. Moreover, the instruments 1400 and the imaging device are positioned and manipulated through incisions or natural orifices in the patient P in a manner such that a center of motion remote from the manipulator and typically located at a position along the instrument shaft is maintained at the incision or orifice by either kinematic mechanical or software constraints.

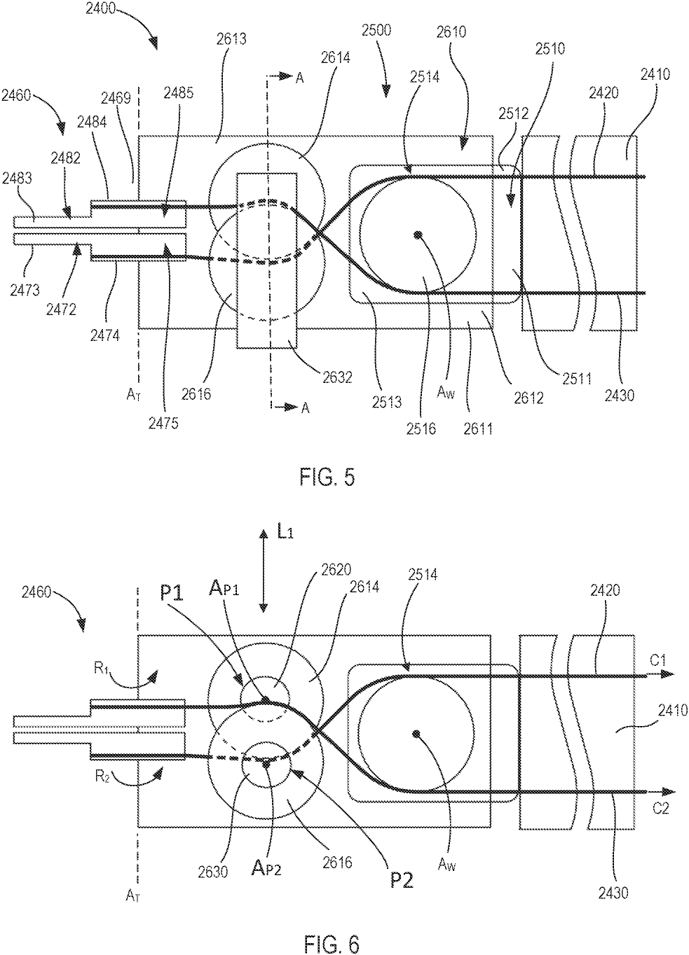

FIGS. 5-7 are schematic illustrations of a portion of a medical instrument 2400 according to an embodiment. In some embodiments, the medical instrument 2400 or any of the components therein are optionally parts of a surgical system that performs surgical procedures, and which can include a manipulator unit, a series of kinematic linkages, a series of cannulas, or the like. The medical instrument 2400 includes a shaft 2410, a first cable 2420 (which acts as a first tension member), a second cable 2430 (which acts as a second tension member), an end effector 2460, and a wrist assembly 2500 (see FIG. 5). The wrist assembly 2500 includes a first link 2510 and a second link 2610. The first link 2510 includes a first link body 2511 having a proximal portion 2512 and a distal portion 2513. The second link 2610 includes a second link body 2611 having a proximal portion 2612 and a distal portion 2613. The proximal portion 2512 of the first link 2510 is coupled to the shaft 2410. The proximal portion 2612 of the second link 2610 is rotatably coupled to the distal portion 2513 of the first link 2510 such that the second link 2610 is operable to rotate relative to the first link 2510 about the rotation axis A_{W} (see FIG. 6 where rotation axis A_{W} extends out of the page). The end effector 2460 is rotatably coupled to the distal portion 2613 of the second link 2610.

The end effector 2460 includes a first tool member 2472 and a second tool member 2482. The first tool member 2472 and the second tool member 2482 are each configured to rotate relative to the wrist assembly 2500 and each other about a rotation axis A_{T} to engage or manipulate a target tissue during a surgical procedure. For example, in some embodiments, one or both of the first tool member 2472 and the second tool member 2482 can include an engagement surface that functions as a gripper, cutter, tissue manipulator, or the like. In other embodiments, one or both of the first tool member 2472 and the second tool member 2482 can be an energized tool member that is used for cauterization or electrosurgical procedures. The first tool member 2472 is coupled to the first cable 2420 such that a tension force exerted by the first cable 2420 on the first tool member 2472 produces a rotational torque about the rotation axis A_{T}. Similarly, the second tool member 2482 is coupled to the second cable 2430 such that a tension force exerted by the second cable 2430 on the second tool member 2482 produces a rotational torque about the rotation axis A_{T}. The end effector 2460 can be operatively coupled to the wrist assembly 2500 such that the end effector 2460 (and the tool members) rotates about an axis of rotation A_{T}. For example, movement of the first cable 2420 causes the first tool member 2472 to rotate about the rotation axis A_{T}. Movement of the second cable 2430 causes the second tool member 2482 to rotate about the rotation axis A_{T}. In this manner, the first tool member 2472 and the second tool member 2482 can be actuated to engage or manipulate a target tissue during a surgical procedure.

As shown in FIG. 6, the first cable 2420 and the second cable 2430 can each be routed between a proximal mechanical structure (not shown), the shaft 2410, the wrist assembly 2500, and the end effector 2460. For example, the first cable 2420 may be coupled to a capstan or pull-pull mechanism within a proximal mechanical structure to pull the first cable 2420 in the direction C₁ to cause the first tool member 2472 to rotate about the rotation axis A_{T} in the direction R₁. In some embodiments, the capstan or pull-pull mechanism may feed the first cable 2420 in the direction opposite to the direction C₁ in response to a rotation of the first tool member 2472 about the rotation axis A_{T} in the direction opposite to R₁. Similarly, the second cable 2430 may be coupled to a capstan or pull-pull mechanism of the proximal mechanical structure to pull the second cable 2430 in the direction C₂ to cause the second tool member 2482 to rotate about the rotation axis A_{T} in the direction R₂. In some embodiments, the capstan may feed the second cable 2430 in the direction opposite to the direction C₂ in response to a rotation of the second tool member 2482 about the rotation axis A_{T} in the direction opposite to R₂.

As shown in FIGS. 5 and 6, the wrist assembly 2500 includes a guide member 2514 and a set of idler pulleys 2614, 2616. The guide member 2514 and the idler pulleys 2614, 2616 include surfaces about which the first cable 2420 and the second cable 2430 are at least partially wrapped to route the cables through the wrist assembly 2500 and to the end effector 2460. As described in more detail herein, the locations of the guide member 2514 and the idler pulleys 2614, 2616 are configured to produce the desired torque on the tool members while also providing the desired the cable life. For example, cable life can be improved by adjusting several different design parameters, including increasing the diameter of the idler pulleys, increasing the cable diameter, and increasing the ratio of the pulley diameter to the cable diameter. Some of these design parameters, however, can be mutually exclusive (e.g., increasing the cable size will decrease the ratio of the pulley diameter to the cable diameter) or may result undesired adjustment of other parameters (e.g., a change in the fleet angle where the cable is coupled to the tool member). Adjusting certain design parameters to enhance cable life may also be incompatible with producing a smaller tool size. For example, increasing the pulley diameter can lead to lower friction, reduced bending stresses and improved cable life (i.e., a greater number of cycles through which the cable can be tensioned), but can also result in an increase in the overall tool size. Thus, as described below, the idler pulleys 2614, 2616 can be coupled to the wrist assembly 2500 in an overlapping manner such that a rotation axis A_{P2} of the outer idler pulley 2616 extends outside of an outer perimeter of the inner idler pulley 2614. Stated differently, the inner idler pulley 2614 defines the outer perimeter of the inner idler pulley 2614 and the pulley axis of the outer idler pulley 2616 is outside a projection of the outer perimeter of the inner idler pulley 2614. In this manner, the wrist assembly 2500 can accommodate larger pulleys within the desired instrument size. In some embodiments, the rotation axis A_{P1} is parallel with the rotation axis A_{P2}.

The guide member 2514 is supported on the proximal portion 2612 of the second link 2610 and provides one or more surfaces to route the first cable 2420 and the second cable 2430 through the wrist assembly 2500 and to the set of idler pulley 2614, 2616. In some embodiments, the guide member can be a fixed structure, similar to the thimble structures shown and described in U.S. Pat. Publ. No. 2020/0390430 (filed Aug. 21, 2020), entitled “Low-Friction, Small Profile Medical Tools Having Easy-to Assemble Components,” which is incorporated herein by reference in its entirety. Such fixed structures can include any suitable low friction coating or surface treatment to reduce cable friction. In other embodiments, the guide member 2514 can include one or more idler pulleys (which function as a proximal set of idler pulleys) about which the first cable 2420 and/or the second cable 2430 can rotate. In such embodiments, the proximal set of idler pulleys rotate about the rotation axis A_{W}. In other words, the rotational axes of an inner idler pulley and an outer idler pulley are co-axial.

The set of idler pulleys includes an inner idler pulley 2614 (i.e., the idler pulley 2614 is coupled on an inboard side of the second link 2610; closer to a central axis of the second link 2610) and an outer pulley 2616 (i.e., the idler pulley 2616 is coupled on an outboard side of the second link 2610; further away from the central axis of the second link 2610). Similarly stated, the inner pulley 2614 is a first distance d_{P1} from the second link 2610 and the outer pulley 2616 is a second distance d_{P2} from the second link 2610, with the second distance d_{P2} being greater than the first distance d_{P1}.

The second link 2610 includes a first support member 2620 that extends outwardly from a second link body 2611. The inner idler pulley 2614 is rotatably supported on the first support member 2620 to rotate about rotation axis A_{P1}. In some embodiments, the first support member 2620 is a pin or a boss coupled to the second link 2610. In some embodiments, the first support member 2620 is formed together with, or is monolithic with the second link body 2611. In some embodiments, a second support member 2630 is supported by a support bracket 2632 and extends toward the second link body 2611. The outer idler pulley 2616 is rotatably supported on the second support member 2630 to rotate about rotation axis A_{P2}. This arrangement allows the rotation axis A_{P1} to be offset from rotation axis A_{P2} by an amount such that the rotation axis A_{P1} is outside of an outer perimeter of the second support member 2630 and the rotation axis A_{P2} is outside of an outer perimeter of the first support member 2620, as described below. In this embodiment, the rotation axis A_{P2} is parallel with the rotation axis A_{P1}, but in other embodiments, the rotation axis A_{P2} can be nonparallel with the rotation axis A_{P1}.

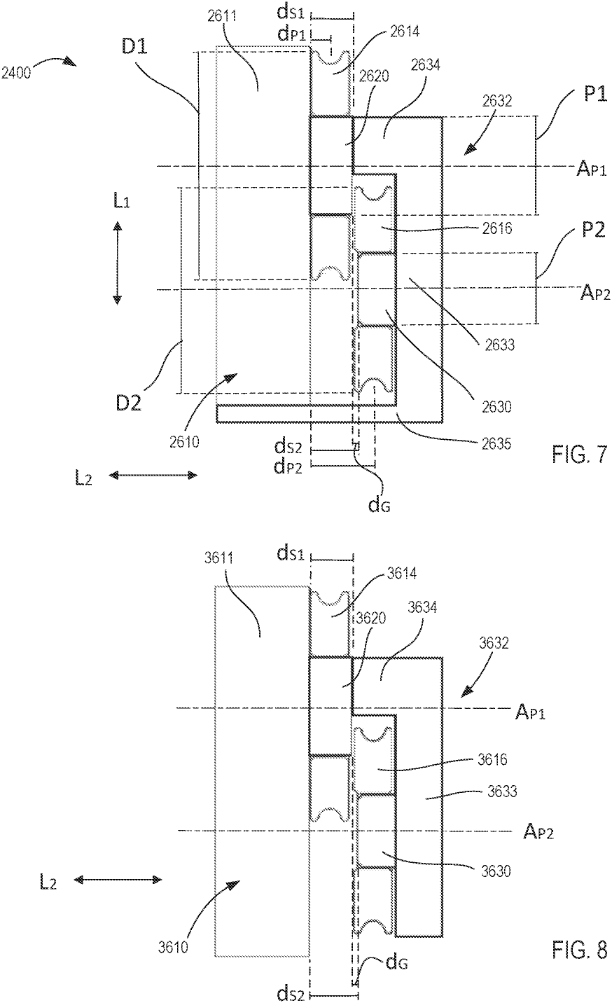

In some embodiments, the support bracket 2632 includes a body portion 2633, a first mounting portion 2634, and a second mounting portion 2635 to secure the second support member 2630 to the second link 2610. The second support member 2630 is attached to the body portion 2633 of the support bracket 2632. In some embodiments, the second support member 2630 and the body portion 2633 are monolithically constructed. The first mounting portion 2634 extends between the first support member 2620 and the body portion 2633 of the support bracket 2632. The second mounting portion 2635 extends between the second link 2610 and the body portion 2633 of the support bracket 2632. Although shown as including a first mounting portion 2634 and a second mounting portion 2635, in other embodiments, the support bracket 2632 can include any suitable structure to facilitate the arrangement of the second support member 2630 extending towards the second link body 2611. In some embodiments, the support bracket 2632 does not include the first mounting portion 2634, rather the first support member 2620 extends from the body portion 2633, which is in turn supported by the second mounting portion 2635. In other embodiments, the support bracket 2632 does not include the second mounting portion 2635, rather the body portion 2633 is coupled to the first support member 2620 via the first mounting portion 2634. As shown in FIG. 7, the set of idler pulleys 2614, 2616 are at least partially enclosed between the second link 2610 and the body portion 2633 to prevent the rotating elements of the set of idler pulleys 2614, 2616 from contacting a surgical site.

As shown best in FIG. 6 the first support member 2620 defines an outer perimeter P1 (i.e., outer circumferential perimeter) and the second support member 2630 defines an outer perimeter P2 (i.e., outer circumferential perimeter). A projection of the outer perimeter P1 of the first support member 2620 parallel with the rotation axis A_{P1} extends outside of the outer perimeter of the second support member 2630. As shown in FIG. 7, the inner idler pulley 2614 includes an outer diameter D1 (i.e., outer circumferential perimeter of the inner idler pulley 2614) and the outer idler pulley 2616 includes an outer diameter D2 (i.e., outer circumferential perimeter of the outer idler pulley 2616). A projection of the outer perimeter of the outer idler pulley 2616 parallel with the rotation axis A_{P2} overlaps with the outer perimeter of the inner idler pulley 2614. In some embodiments, the rotation axis A_{P2} is outside a projection of the outer diameter D1 of the inner idler pulley 2614. Additionally, in some embodiments, the rotation axis A_{P2} does not intersect the first support member 2620. In other words, the rotation axis A_{P2} does not extend through any portion of the first support member 2620.

The inner idler pulley 2614 and the outer idler pulley 2616 are laterally spaced in a first direction L₁ (see FIGS. 6 and 7), which is parallel to the rotation axis A_{T}, and in a second direction L₂ (see FIG. 7) which is parallel to the rotation axis A_{P2}._{.} As shown in FIG. 7, the first support member 2620 extends a first distance d_{S1} away from the second link 2610. The second support member 2630 is spaced away from the second link 2610 a second distance d_{S2}, the second distance d_{S2} being greater than the first distance first distance d_{S1}. The first support member 2620 is spaced a gap distance do from the second support member 2630 in a second direction L₂. In this manner, the set of idler pulleys 2614, 2616 can be sized large enough to provide a gradual transition from the guide member 2514 to the first and second drive pulleys 2474, 2484. Additionally, by having the set of idler pulleys 2614, 2616 partially overlap as opposed to being positioned completely side-by-side in the first direction L₁, the overall height of the wrist assembly 2500 (in the direction of the rotation axis A_{T}) can be reduced. In some embodiments, the set of idler pulleys 2614, 2616 are positioned relative to one another such that second link 2610 and the wrist assembly 2500 can be inserted through a cannula with an inner diameter equal to or smaller than 8.5 mm (and more specifically between about 4.0 mm and 8.5 mm, or between about 4.0 mm and 6.0 mm). In some embodiments, the second set of idler pulley 2614, 2616 are positioned relative to one another such that second link 2610 and the wrist assembly 2500 can be inserted through a cannula with an inner diameter of about 5.0 mm.

Although the support bracket 2632 of FIGS. 5 and 7 is shown as including a first mounting portion 2634 and a second mounting portion 2635, FIG. 8 depicts a support bracket 3632 including a body portion 3633 and a single mounting portion 3634 according to an embodiment. The mounting portion 3634 is coupled to the first support member 3620. Because the body portion 3633 is supported only on one end via the mounting portion 3634 coupled to the first support member 3620, the body portion 3633 is cantilevered.

Similar to the first support member 2620, the first support member 3620 extends from a second link body 3611 of a second link 3610. An inner idler pulley 3614 is rotatably supported on the first support member 3620 to rotate about rotation axis A_{P1}. The support bracket 3632 includes a second support member 3630, the second support member 3630 extending from the body portion 3633 towards the second link body 3611.

As shown in FIG. 8, the first support member 3620 extends a first distance d_{S1} away from the second link 3610. The second support member 3630 is spaced away from the second link 3610 a second distance d_{S2}, the second distance d_{S2} being greater than the first distance first distance d_{S1}. The first support member 3620 is spaced a gap distance do from the second support member 3630 in a second direction L₂.

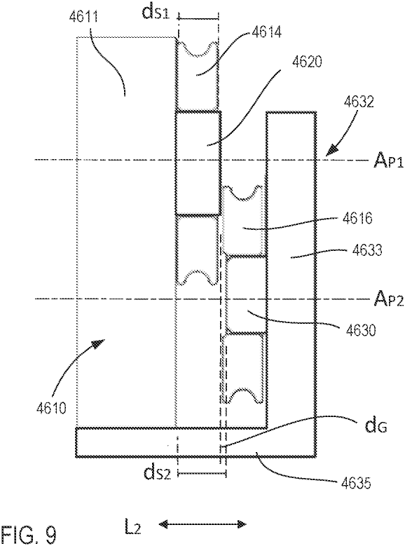

FIG. 9 depicts a support bracket 4632 including a body portion 4633 and a single mounting portion 4635. The mounting portion 4635 is coupled to the first support member 4620. Because the body portion 4633 is supported only on one end via the mounting portion 4635 coupled to the second link body 4611, the body portion 4633 is cantilevered.

Similar to the first support member 2620, the first support member 4620 extends from a second link body 4611 of a second link 4610. An inner idler pulley 4614 is rotatably supported on the first support member 4620 to rotate about rotation axis A_{P1}. The support bracket 4632 includes a second support member 4630, the second support member 4630 extending from the body portion 4633 towards the second link body 4611. In this manner, the second support member 4630 is coupled to the second link body 4611 independent of the first support member 4620

As shown in FIG. 9, the first support member 4620 extends a first distance d_{S1} away from the second link 4610. The second support member 4630 is spaced away from the second link 4610 a second distance d_{S2}, the second distance d_{S2} being greater than the first distance first distance d_{S1}. The first support member 4620 is spaced a gap distance d_{G} from the second support member 4630 in a second direction L₂.

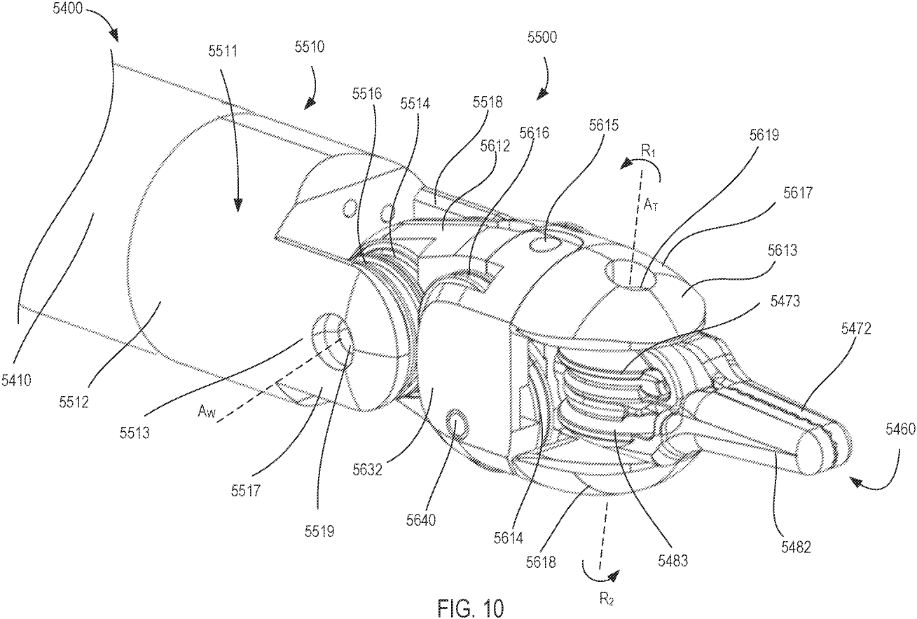

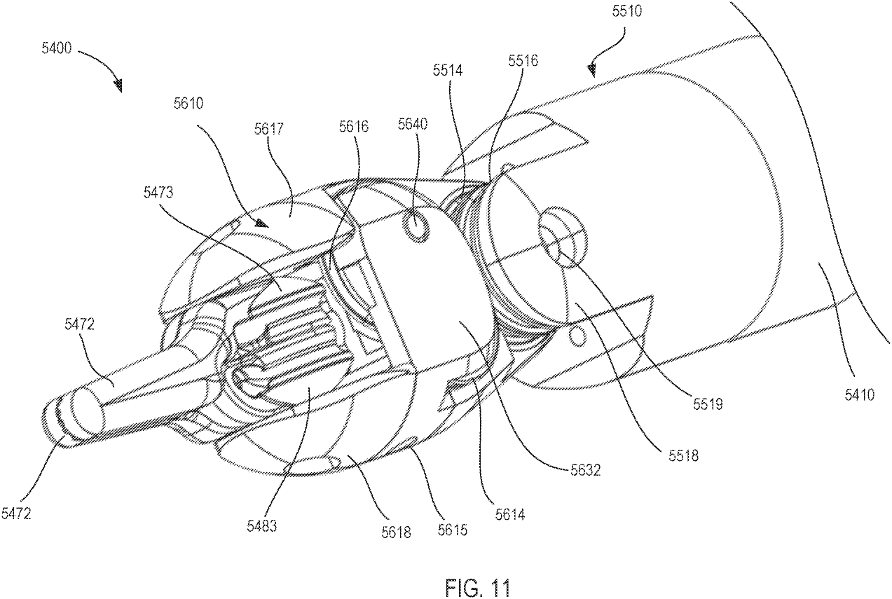

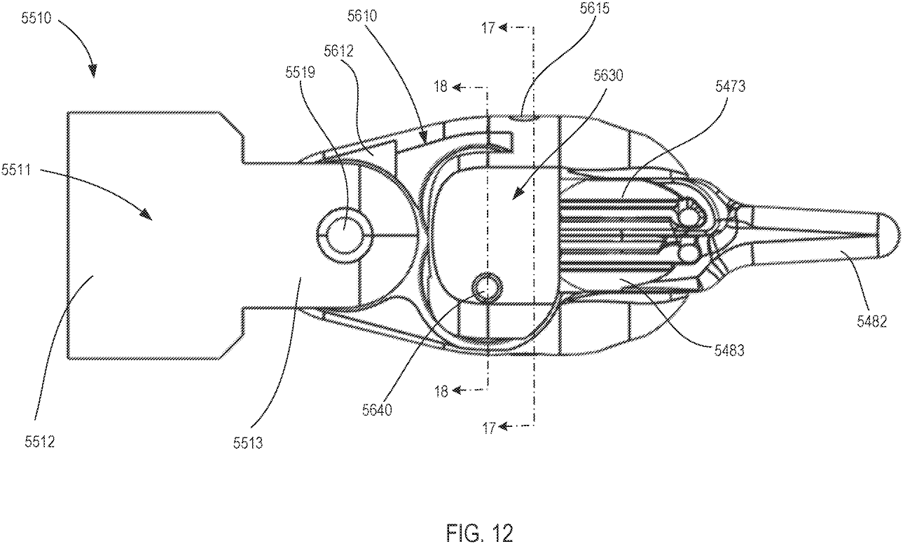

FIGS. 10-21 are various views of a medical instrument 5400, according to an embodiment. In some embodiments, the instrument 5400 or any of the components therein are optionally parts of a surgical system that performs surgical procedures, and which can include a manipulator unit, series of kinematic linkages, a series of cannulas, or the like. The instrument 5400 (and any of the instruments described herein) can be used in any suitable surgical system, such as the MIRS system 1000 shown and described above. The instrument 5400 includes a proximal mechanical structure (not shown), a shaft 5410, a first cable 5420 (which acts as a first tension member), a second cable 5430 (which acts as a second tension member), a distal wrist assembly 5500, and a tool end effector 5460. The wrist assembly 5500 includes a first link 5510 and a second link 5610. The first link 5510 includes a first link body 5511 having a proximal portion 5512 and a distal portion 5513. The distal portion 5513 of the first link 5510 includes a connector with clevis ears 5517, 5518.

The second link 5610 includes a second link body 5611, a proximal portion 5612 and a distal portion 5613. The proximal portion 5512 of the first link 5510 is coupled to the shaft 5410. The proximal portion 5612 of the second link 5610 is rotatably coupled to the clevis ears 5517, 5518 of the first link 5510 such that the second link 5610 is operable to rotate relative to the first link 5510 about the rotation axis A_{W}. In some embodiments, the proximal portion 5612 of the second link 5610 is rotatably coupled to the clevis ears 5517, 5518 via a pin 5519. The end effector 5460 is rotatably coupled to clevis ears 5617, 5618 of the second link 5610 such that the end effector 5460 is operable to rotate relative to the second link 5610 about the rotation axis A_{T}. The end effector 5460 is rotatably coupled to the clevis ears 5617, 5618 of the second link 5610 via a pin 5619.

The end effector 5460 includes a first tool member 5472 and a second tool member 5482. The instrument 5400 is configured such that movement of the first cable 5420 and second cable 5430 produces rotation of the end effector 5460 about a first axis of rotation A_{T} (see FIG. 10, which functions as a yaw axis, the term yaw is arbitrary), rotation of the wrist assembly 5500 about a second axis of rotation A_{W} (see FIG. 10 which functions as a pitch axis, the term pitch is arbitrary), a gripping rotation of the tool end effector 5460 about the first axis of rotation A_{T}, or any combination of these movements. For example, in some embodiments, one or both of the first tool member 5472 and the second tool member 5482 can include an engagement surface that functions as a gripper, cutter, tissue manipulator, or the like. In some embodiments, one or both of the first tool member 5472 and the second tool member 5482 can be an energized tool member that is used for cauterization or electrosurgical procedures.

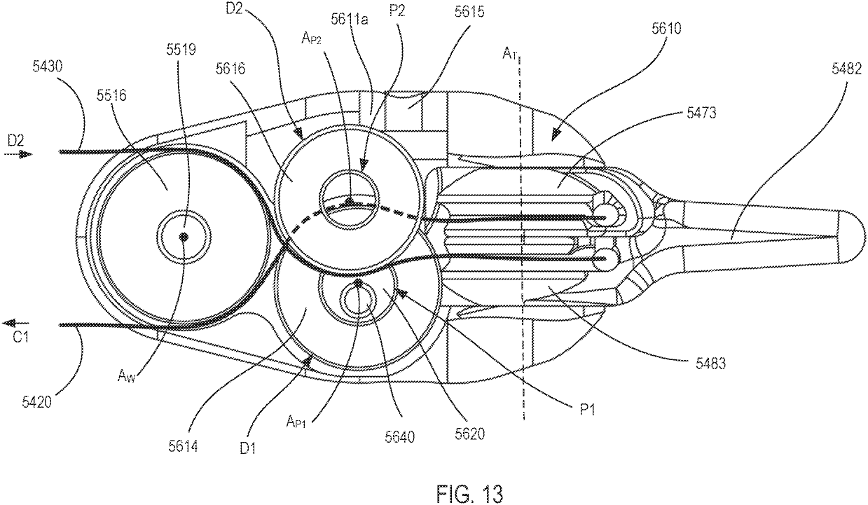

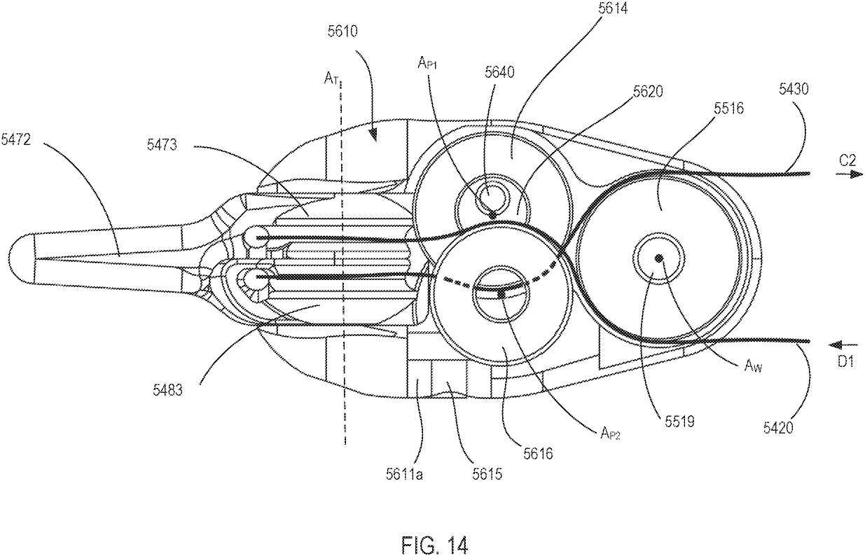

The first tool member 5472 includes a driven pulley 5473 and the first cable 5420 engages the driven pulley 5473 such that a tension force in a direction C₁ exerted by the first cable 5420 on the first tool member 5472 produces a rotational torque about the rotation axis A_{T} (see FIGS. 10 and 13). Similarly, the second tool member 5482 includes a pulley 5483 coupled to the second cable 5430 such that a tension force in a direction C₂ exerted by the second cable 5430 on the second tool member 5482 produces a rotational torque about the rotation axis A_{T} (see FIGS. 10 and 14). Similarly stated, movement of the first cable 5420 causes the first tool member 5472 to rotate about the rotation axis A_{T}. Movement of the second cable 5430 causes the second tool member 5482 to rotate about the rotation axis A_{T}. In this manner, the first tool member 5472 and the second tool member 5482 can be actuated to engage or manipulate a target tissue during a surgical procedure.

As shown in FIGS. 13 and 14, the first cable 5420 and the second cable 5430 can each be routed between a proximal mechanical structure (not shown), the shaft 5410, the wrist assembly 5500, and the end effector 5460. For example, the first cable 5420 may be coupled to a capstan or pull-pull mechanism within a proximal mechanical structure to pull the first cable 5420 in the direction C₁ to cause the first tool member 5472 to rotate about the rotation axis A_{T} in the direction R₁. In some embodiments, the capstan or pull-pull mechanism may feed the first cable 5420 in the direction opposite to the direction C₁ in response to a rotation of the first tool member 5472 about the rotation axis A_{T} in the direction opposite to R₁. The second cable 5430 may be coupled to a capstan or pull-pull mechanism of the proximal mechanical structure to pull the second cable 5430 in the direction C₂ to cause the second tool member 5482 to rotate about the rotation axis A_{T} in the direction R₂. In some embodiments, the capstan may feed the second cable 5430 in the direction opposite to the direction C₂ in response to a rotation of the second tool member 5482 about the rotation axis A_{T} in the direction opposite to R₂.

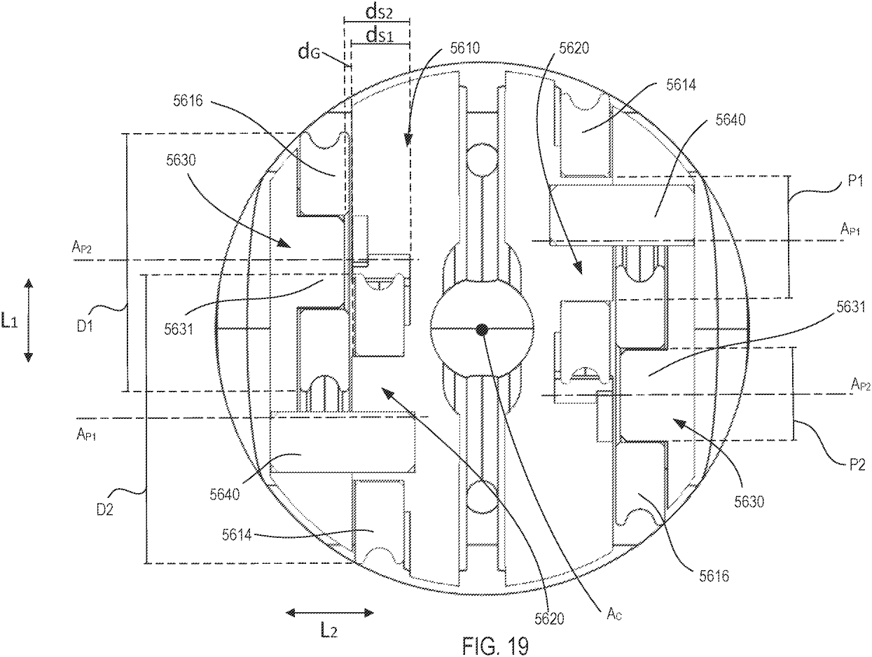

As shown in FIGS. 13-15, the wrist assembly 5500 includes proximal inner idler pulleys 5514, proximal outer idler pulleys 5516, distal inner idler pulleys 5614, and distal outer idler pulleys 5616. The idler pulleys 5514, 5516, 5614, 5616 each include surfaces about which the first cable 5420 and the second cable 5430 are at least partially wrapped to route the cables through the wrist assembly 5500 and to the end effector 5460. In some embodiments, the arrangement of the idler pulleys 5514, 5516, 5614, 5616 on the front side of the wrist assembly 5500 (see FIG. 13 the identification of the front side of the wrist assembly 5500 being arbitrary) can be the same as the arrangement of the idler pulleys 5514, 5516, 5614, 5616 on the rear side of the wrist assembly 5500 except that the components are revolved 180 degrees about a central axis A_{C} of the wrist assembly 5500 (see FIGS. 14 and 19). For brevity, the arrangement of the idler pulleys 5514, 5516, 5614, 5616 and routing of the cables 5420, 5430 will be described in greater detail with respect to the front side of the of the wrist assembly 5500 (see FIGS. 10, 12, 13, 15, 16, 17, and 18). However, the rear side of the wrist assembly (see FIGS. 11 and 14) can include the same or a similar arrangement of the idler pulleys 5514, 5516, 5614, 5616 and routing of the cables 5420, 5430.

As described in more detail herein, the location of the proximal inner idler pulleys 5514, proximal outer idler pulleys 5516, distal inner idler pulleys 5614, and distal outer idler pulleys 5616 is configured to produce the desired torque on the first tool member 5472 and a second tool member 5482 while also providing the desired the cable life.

For example, cable life can be improved by adjusting several different design parameters, including increasing the diameter of the idler pulleys, increasing the cable diameter, and increasing the ratio of the pulley diameter to the cable diameter. Some of these design parameters, however, can be mutually exclusive (e.g., increasing the cable size will decrease the ratio of the pulley diameter to the cable diameter) or may result undesired adjustment of other parameters (e.g., a change in the fleet angle where the cable is coupled to the tool member). Adjusting certain design parameters to enhance cable life may also be incompatible with producing a smaller tool size. For example, increasing the pulley diameter can lead to lower friction, reduced bending stresses and improved cable life, but can also result in an increase in the overall tool size. Thus, as described below, the idler pulleys 5614, 5616 can be coupled to the wrist assembly 5500 in an overlapping manner such that a rotation axis A_{P2} of the distal outer idler pulley 5616 extends outside of an outer perimeter of the distal inner idler pulley 5614 or the support member 5620. Stated differently, the distal inner idler pulley 5614 defines the outer perimeter and the rotation axis A_{P2} of the distal outer idler pulley 5616 is outside a projection of the outer perimeter P1 of the support member 5620 (see FIG. 19). In this manner, the wrist assembly 5500 can accommodate larger pulleys within the desired instrument size (see FIG. 19). In some embodiments, the rotation axis A_{P1} is parallel with the rotation axis A_{P2}.

In some embodiments, an outer diameter of the distal outer idler pulley 5616 is greater than an outer diameter of the distal inner idler pulley 5614. In some embodiments, the distal inner idler pulley 5614 has an outer diameter of between about 3 mm and about 3.7 mm, and more particularly about 3.683 mm (0.145 inches). In some embodiments, the distal outer idler pulley 5616 has an outer diameter of between about 3.4 mm and about 4.2 mm, and more particularly about 4.191 mm (0.165 inches). In some embodiments, the proximal idlers pulleys 5514, 5516 have an outer diameter of between about 3.0 mm and 4.2 mm, and more particularly about 4.242 mm (0.167 inches). In some embodiments, an outer diameter of the first and second cables 5420, 5430 is between about 0.457 mm (0.018 inches) to about 0.635 mm (0.025 inches). In some embodiments, a ratio of a diameter of the distal inner idler pulley 5614 to an outer diameter of the first cable 5420 is greater than about 6.5. In some embodiments, a ratio of a diameter of the distal inner idler pulley 5614 to an outer diameter of the first cable 5420 is between about 6.5 to 9.2.

The proximal inner idler pulleys 5514 and proximal outer idler pulleys 5516 are supported on the pin 5519 to rotate about the rotation axis A_{W}. The proximal inner idler pulleys 5514 and the proximal outer idler pulleys 5516 each include surfaces about which the first cable 5420 and the second cable 5430 are at least partially wrapped to route the cables through the wrist assembly 5500 and to the set of idler pulley 5614, 5616. Although the proximal inner idler pulleys 5514 and proximal outer idler pulleys 5516 are shown as being arranged coaxially, in some embodiments, the rotational axis of the proximal inner idler pulleys 5514 and the rotational axis of the proximal outer idler pulleys 5516 are non-coaxial and/or non-parallel.

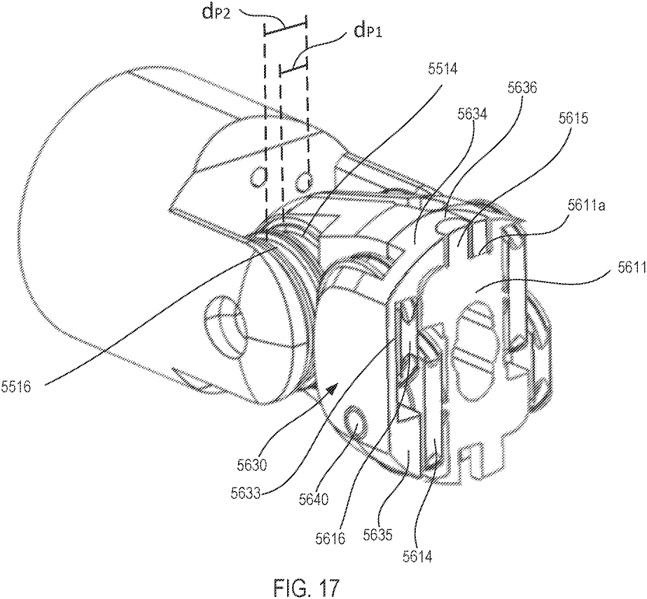

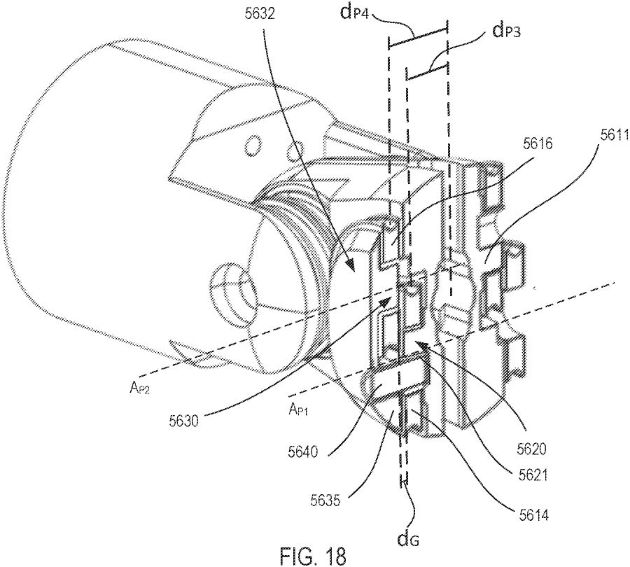

The proximal inner idler pulleys 5514 are coupled on an inboard side of the second link 5610 (i.e., closer to a central axis of the second link 5610) and the proximal outer idler pulleys 5516 are coupled on an outboard side of the second link 5610 (i.e., further away from the central axis of the second link 5610). Similarly stated, the proximal inner idler pulleys 5514 is a first distance d_{P1} from the second link 5610 and the proximal outer idler pulleys 5516 is a second distance d_{P2} from the second link 5610, with the second distance d_{P2} being greater than the first distance d_{P1} (see FIG. 17). The distal inner idler pulleys 5614 are coupled on an inboard side of the second link 5610 (i.e., closer to a central axis of the second link 5610) and the distal outer idler pulleys 5616 are coupled on an outboard side of the second link 5610 (i.e., further away from the central axis of the second link 5610). Similarly stated, the distal inner idler pulleys 5614 is a third distance d_{P3} from the second link 5610 and the distal outer idler pulleys 5616 is a fourth distance d_{P4} from the second link 5610, with the fourth distance d_{P1} being greater than the third distance d_{P3} (see FIG. 18). In some embodiments, the first distance d_{P1} of the proximal inner idler pulleys 5514 is equal to the third distance d_{P3} as the distal inner idler pulleys 5614. In some embodiments, the second distance d_{P2} of the proximal outer idler pulleys 5516 is equal to the fourth distance d_{P4} as the distal outer idler pulleys 5616.

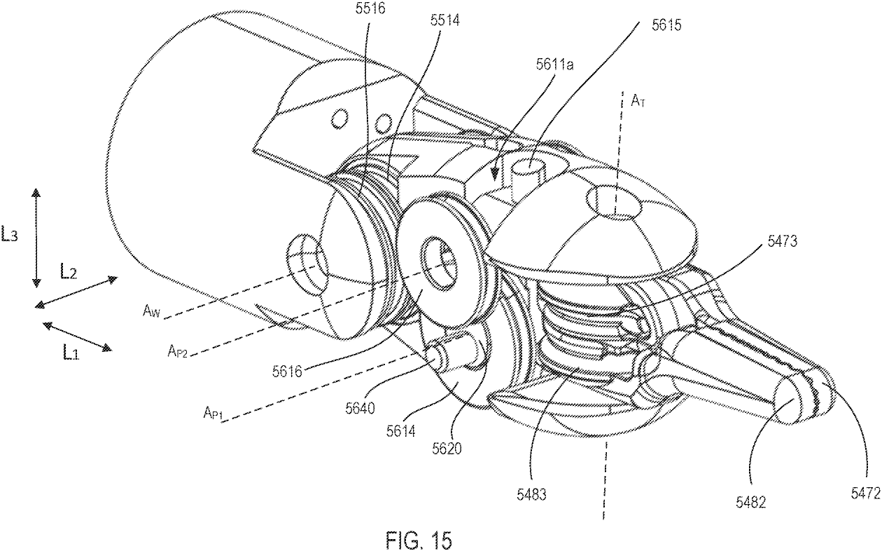

The second link 5610 includes a first support member 5620 that extends outwardly from a second link body 5611. The distal inner idler pulley 5614 is rotatably supported on the first support member 5620 to rotate about rotation axis A_{P1} (see, e.g., FIGS. 15 and 18). In some embodiments, the first support member 5620 is a pin or a boss coupled to the second link 5610. In some embodiments, the first support member 5620 is formed together with, or is monolithic with the second link body 5611. The second link 5610 also includes a second support member 5630 that extends toward the second link body 5611. The distal outer idler pulley 5616 is rotatably supported on a pin or boss 5631 of the second support member 5630 to rotate about rotation axis A_{P2}. This arrangement allows the rotation axis A_{P1} to be offset from rotation axis A_{P2} by an amount such that the rotation axis A_{P1} is outside of an outer perimeter of the second support member 5630 and the rotation axis A_{P2} is outside of an outer perimeter of the first support member 5620, as described below. In this embodiment, the rotation axis A_{P2} is parallel with the rotation axis A_{P1}, but in other embodiments, the rotation axis A_{P2} can be nonparallel with the rotation axis A_{P1}. A_{P1} and A_{P2} can be non parallel to axis A_{W} in some embodiments.

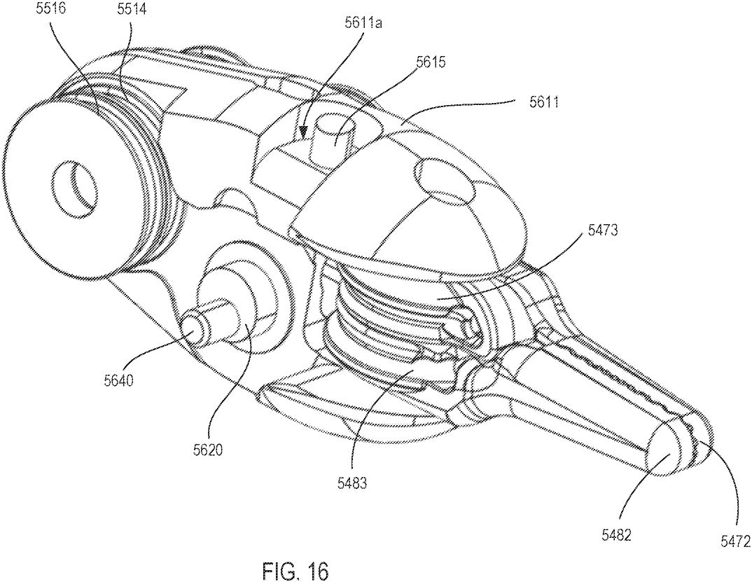

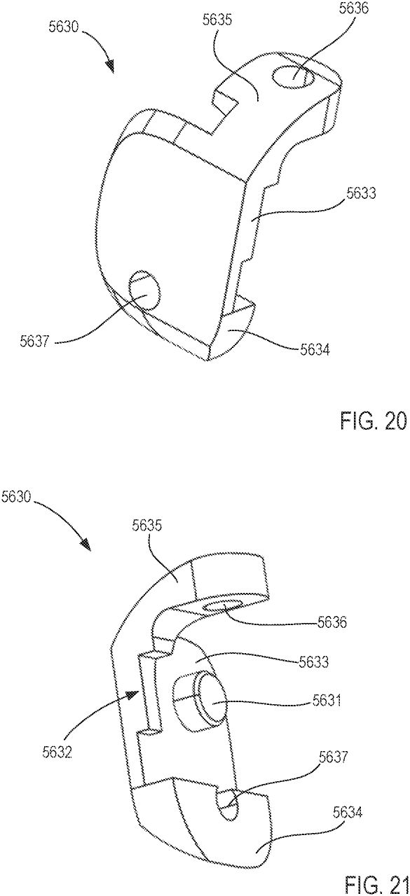

As shown in FIGS. 20 and 21, the second support member 5630 includes a support bracket 5632. The support bracket 5632 includes a body portion 5633, a first mounting portion 5634, and a second mounting portion 5635 to secure the second support member 5630 to the second link 5610. The second support member 5630 is attached to the body portion 5633 of the support bracket 5632. In some embodiments, the second support member 5630 and the body portion 5633 are monolithically constructed. The first mounting portion 5634 extends between the first support member 5620 and the body portion 5633 of the support bracket 5632. The first mounting portion 5634 includes a mounting hole 5636 for receiving a mounting boss 5615 of the second link 5610. The second link body 5611 includes a recessed surface 5611a (see FIGS. 15 and 16) configured to receive the first mounting portion 5634 such that an outer surface of the first mounting portion 5634 is flush with an outer surface of the second link body 5611 when seated within the recessed surface 5611a (see FIGS. 10-12). In some embodiments, the mounting boss 5615 and the recessed surface 5611a are formed by machining an outer surface of the second link body 5611. In some embodiments, the mounting boss 5615 and the recessed surface 5611a are formed during casting or molding of the second link body 5611. Although the mounting hole 5636 is shown as being a circular through-hole, in other embodiments, the mounting hole 5636 is a non-circular through-hole or a hole that extends only partially through the first mounting portion 5634 (e.g., blind hole). In some embodiments, the first mounting portion 5634 is secured to the second link 5610 via a fastener (e.g., screw, bolt, or the like). In other embodiments, the first mounting portion 5634 is secured to the second link 5610 via interference fit, welding, and/or adhesives.

The second mounting portion 5635 of the second support member 5630 extends between the second link 5610 and the body portion 5633 of the support bracket 5632. The second mounting portion 5635 includes a mounting hole 5637 for receiving a mounting pin 5640 to secure the second mounting portion 5635 to the first support member 5620. As shown in FIG. 18, the first support member 5620 includes a blind hole 5621 for receiving the mounting pin 5640. In some embodiments, the second mounting portion 5635 is secured to the first support member 5620 via a fastener (e.g., screw, bolt, or the like). In other embodiments, the second mounting portion 5635 is secured to the first support member 5620 via interference fit, welding, and/or adhesives.

As shown in FIGS. 10-12, the set of distal idler pulleys 5614, 5616 are at least partially enclosed between the second link 5610 and the body portion 5633 of the second support member 5630 to prevent the rotating elements of the set of distal idler pulleys 5614, 5616 from contacting a surgical site.

As shown best in FIGS. 13 and 19, the first support member 5620 includes an outer perimeter P1 (i.e., outer circumferential perimeter) and the second support member 2630 includes an outer perimeter P2 (i.e., outer circumferential perimeter). A projection of the outer perimeter P1 of the first support member 5620 parallel with the rotation axis A_{P1} extends outside of the outer perimeter of the second support member 5630. As shown in FIG. 13, the distal inner idler pulley 5614 includes an outer diameter D1 (i.e., outer circumferential perimeter) and the distal outer idler pulley 5616 includes an outer diameter D2 (i.e., outer circumferential perimeter). A projection of the outer perimeter of the distal outer idler pulley 5616 parallel with the rotation axis A_{P2} overlaps with the outer diameter D2 of the distal inner idler pulley 5614. The rotation axis A_{P2} is outside a projection of the outer diameter D1 of the distal inner idler pulley 5614. Additionally, the rotation axis A_{P2} does not intersect the first support member 5620. In other words, the rotation axis A_{P2} does not extend through any portion of the first support member 5620.

The distal inner idler pulley 5614 and the distal outer idler pulley 5616 are laterally spaced in a first direction L₁, which is parallel to the rotation axis A_{T}, and in a second direction L₂, which is parallel to the rotation axis A_{P2} (see FIGS. 15 and 19). As shown in FIG. 19, the first support member 5620 extends a first distance d_{S1} away from the second link 5610. The second support member 5630 is spaced away from the second link 5610 a second distance d_{S2}, the second distance d_{S2} being greater than the first distance first distance dsi. In some embodiments, the first distance d_{S1} is between about 0.75 mm to about 0.95 mm. In some embodiments, the second distance d_{S2} is between about 0.085 mm to about 1.05 mm. The first support member 5620 is spaced a gap distance d_{G} from the second support member 5630 in a second direction L₂. In some embodiments, the gap distance d_{G} is between about 0.05 mm to about 0.015 mm. In this manner, the set of distal idler pulleys 5614, 5616 can be laterally offset and sized large enough to provide a gradual transition from the set of proximal idler pulleys 5514, 5516 to the first and second drive pulleys 5473, 5483.

Additionally, by having the set of distal idler pulleys 5614, 5616 partially overlap as opposed to being positioned completely side-by-side in the first direction L₁, the overall height of the wrist assembly 5500 (in the direction of the rotation axis A_{T}) can be reduced while maintaining sufficient cable life for a multiuse instrument. In some embodiments, the set of distal idler pulleys 5614, 5616 are positioned relative to one another such that second link 5610 and the wrist assembly 5500 can be inserted through a cannula with an inner diameter equal to or smaller than 9.0 mm. In some embodiments, the set of distal idler pulley 5614, 5616 are positioned relative to one another such that second link 5610 and the wrist assembly 5500 can be inserted through a cannula with an inner diameter of about 5.0 mm.

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a series of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the tool members can be constructed from any material, such as medical grade stainless steel, nickel alloys, titanium alloys or the like. Further, any of the links, tool members, tension members, or components described herein can be constructed from multiple pieces that are later joined together. For example, in some embodiments, a link can be constructed by joining together separately constructed components. In other embodiments however, any of the links, tool members, tension members, or components described herein can be monolithically constructed.

Although the instruments are generally shown as having an axis of rotation of the tool members (e.g., axis A_{T}) that is normal to an axis of rotation of the wrist member (e.g., axis A_{R}), in other embodiments any of the instruments described herein can include a tool member axis of rotation that is offset from the axis of rotation of the wrist assembly by any suitable angle.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

What is claimed is:

1. A medical device, comprising:
a wrist link comprising a wrist link body and an inner pulley support extending outward from the wrist link body;
an inner pulley rotatably mounted on the inner pulley support to rotate about an inner pulley axis, the inner pulley support extending a first distance away from the wrist link;
an outer pulley support coupled to the wrist link body and extending spaced from the wrist link body;
an outer pulley rotatably mounted on the outer pulley support to rotate about an outer pulley axis, the outer pulley support being spaced a second distance away from the wrist link, the second distance larger than the first distance;
a first tension member extending around the inner pulley; and
a second tension member extending around the outer pulley.

2. The medical device of claim 1, wherein:
the medical device further comprises an outer pulley support bracket; and
the outer pulley support bracket comprises a mounting portion that extends between the outer pulley support and the wrist link body.

3. The medical device of claim 1, wherein:
the medical device further comprises an outer pulley support bracket; and
the outer pulley support bracket comprises a mounting portion that extends between the outer pulley support and the inner pulley support.

4. The medical device of claim 1, wherein:
the medical device further comprises an outer pulley support bracket;
the outer pulley support bracket comprises a first mounting portion and a second mounting portion;
the first mounting portion extends between the outer pulley support and the wrist link body; and
the second mounting portion extends between the outer pulley support and the inner pulley support.

5. The medical device of claim 1, wherein:
the first tension member is routed about the inner pulley along an inner pulley arc length; and
the second tension member is routed about the outer pulley along an outer pulley arc length smaller than the inner pulley arc length.

6. The medical device of claim 1, wherein:
the outer pulley axis of rotation is parallel to the inner pulley axis of rotation.

7. The medical device of claim 1, wherein:
the inner pulley comprises an outer perimeter;
the outer pulley comprises an outer perimeter; and
a projection of the outer perimeter of the outer pulley parallel to the outer pulley axis of rotation overlaps the outer perimeter of the inner pulley.

8. The medical device of claim 1, wherein:
the inner pulley comprises an outer perimeter; and
the outer pulley axis of rotation is outside a projection of the outer perimeter of the inner pulley parallel to the inner pulley axis of rotation.

9. The medical device of claim 1, wherein:
the outer pulley axis of rotation does not intersect the inner pulley support.

10. The medical device of claim 2, wherein:
the wrist link body comprises a first material;
the outer pulley support bracket comprises a second material; and
the second material is different from the first material.

11. The medical device of claim 2, wherein:
the inner pulley and the outer pulley are enclosed between the wrist link and the outer pulley support bracket.

12. The medical device of claim 1, wherein:
the inner pulley has a circumference defined by an outer radius of the inner pulley, the first tension member routed about a portion of the circumference of the inner pulley;
the first tension member has a cross-sectional radius; and
a ratio of the outer radius of the inner pulley to the cross-sectional radius of the first tension member is between about 6.5 to 9.2.

13. The medical device of claim 1, wherein:
the first and second tension members comprise Tungsten cables.

14. The medical device of claim 1, wherein:
the wrist link is sized to be inserted through a cannula having an inner diameter equal to or smaller than about 8.5 mm.

15. The medical device of claim 1, wherein:
the medical device further comprises a first tool member and a second tool member;
the first and second tool members are rotatably coupled to the wrist link;
the first tension member is coupled to the first tool member; and
the second tension member is coupled to the second tool member.

16. The medical device of claim 2, wherein:
the inner pulley support extends to the outer pulley support bracket.

17. The medical device of claim 16, wherein:
the inner pulley support extends outside of a projection of an outer perimeter of the outer pulley parallel to the outer pulley axis of rotation.

18. The medical device of claim 2, wherein:
the outer pulley support bracket is coupled to the wrist link body in a snap-fit configuration.

19. The medical device of claim 2, wherein:
the outer pulley support bracket is coupled to the wrist link body in a friction fit configuration.

20. The medical device of claim 1, wherein:
the medical device further comprises a teleoperated surgical instrument; and
the teleoperated surgical instrument comprises the wrist link, the inner pulley, the outer pulley, the outer pulley support, the first tension member, and the second tension member.
